(19) United States
(12) Patent Application Publication
(10) Pub. No.: US 2017/0337349 A1
(43) Pub. Date: Nov. 23, 2017

(54) SYSTEM AND METHOD FOR GENERATING HEALTH DATA USING MEASUREMENTS OF WEARABLE DEVICE

(71) Applicant: KONINKLIJKE PHILIPS N.V., EINDHOVEN (NL)

(72) Inventor: John Cronin, Bonita Springs, FL (US)

(21) Appl. No.: 15/528,921

(22) PCT Filed: Nov. 30, 2015
(86) PCT No.: PCT/EP2015/078082

§ 371 Date: May 23, 2017

Related U.S. Application Data
(60) Provisional application No. 62/086,645, filed on Dec. 2, 2014.

(30) Foreign Application Priority Data
May 26, 2015 (EP) 15169206.8

(51) Int. Cl. G06F 19/00; A61B 5/00; A61B 7/00; A61B 5/0205; A61B 7/04; A61B 5/024; A61B 5/08; A61B 5/11; A61B 5/145; A61B 5/01

(52) U.S. Cl. CPC G06F 19/3481; G06F 19/3418; A61B 5/02055; A61B 5/4041; A61B 5/4266; A61B 5/443; A61B 5/4806; A61B 5/4866; A61B 5/4875; A61B 5/6802; A61B 7/003; A61B 7/04; A61B 5/0022; A61B 5/0816; A61B 5/1118; A61B 5/112; A61B 5/14517; A61B 5/14532; G06F 19/3431; A61B 5/01; A61B 2503/10; A61B 2562/0219; A61B 2562/0223; A61B 5/02438; A61B 5/14546

(57) ABSTRACT

A computer-implemented method or system for generating health data is provided. The method includes receiving a sensor data set measured by one or more sensors of a wearable device over an interval of time, the sensor data set indicating a time series profile of a body parameter of a user of the wearable device over the interval of time. The method also includes determining an activity type of the user matching the time series profile; and calculating a value associated with a health metric, wherein the value is calculated based on the activity type.

### FIELD OF INVENTION

The present invention generally relates to performing calculations using sensor measurement data from a wearable device sensor. More specifically, the present invention relates to determining health data of a user using measurement of wearable device.

### DESCRIPTION OF THE RELATED ART

Wearable technology is a new class of electronic systems that can provide data acquisition through a variety of unobtrusive sensors that may be worn by a user. The sensors gather information, for example, about the environment, the user's activity, or the user's health status. However, there are significant challenges related to the coordination, computation, communication, privacy, security, and presentation of the collected data.

Additionally, there are challenges related to power management given the current state of battery technology. Furthermore, analysis of the data is needed to make the data gathered by the sensors useful and relevant to end-users. In some cases, additional sources of information may be used to supplement the data gathered by the sensors. The many challenges that wearable technology presents require new designs in hardware and software.

The advantages of the wearable device include its proximity to the user and consistency of its computations. For example, a number of wearable devices, while worn by the user, constantly and continuously monitor user's data and/or vital signs of the user. Such information can be useful in subsequent analysis of condition and behavior of the user and/or can be used for performing an action necessitated by the measurements.

However, the constant monitoring of the user's data can reduce the flexibility of the measurements that wearable device can perform, which can lead to undesirable conclusions.

### SUMMARY OF THE CLAIMED INVENTION

Some embodiments of an invention are based on recognition that electronic sensors may be coupled to a wearable device to collect and manipulate data regarding one or more detected parameters or conditions. Sensors that sense acceleration, for example, may be used to collect data relating to motion, which can then be manipulated using calculations in the wearable device. The sensor data sensed by the sensor may be stored in memory, and a processor running an algorithm may identify profiles in the data that can be used for determining health metrics of the users.

Some embodiments of an invention are based on realization that determination of a health metric of a user needs to consider an activity type of the user in addition to other body parameters. For example, if the health metric of a user is a number of calories burned, which can be determined based on a number of steps traveled by the user, the method determining the burned calories needs to consider not only a number of steps taken by the user, but also whether the user was running or walking during that time.

Some embodiments are based on another realization that time series profiles of a body parameter of a user of the wearable device measured over an interval of time can be used to determine activity type of the user. As a matter of definitions, a time series is a sequence of successive data points made over the time interval. As used herein, the time series profile is a function of the successive measurements of one or more sensors of the wearable device.

It is an object of some embodiments of the invention to improve an accuracy of a health metric of a user by considering an activity type of the user in calculation of the health metric. It is another object of some embodiment of the invention to determine the activity type and/or a metric method for the activity type of the user based on a time series profile of a body parameter of a user of the wearable device. As used herein, the body parameter can include, but not limited to various vital signs of the user, such as hydration, calories, blood pressure, blood sugar, blood glucose, insulin, body temperature, heat, heat flux, heart rate, weight, sleep, number of steps, velocity, acceleration, vitamin levels, respiratory rate, heart sound, breathing sound, movement speed, skin moisture, sweat detection, sweat composition, or nerve firings of the user.

Accordingly, one embodiment of the invention discloses a computer-implemented method for generating health data. The method includes receiving a sensor data set measured by one or more sensors of a wearable device over an interval of time, the sensor data set indicating a time series profile of a body parameter of a user of the wearable device over the interval of time; determining an activity type of the user matching the time series profile; and calculating a value associated with a health metric, wherein the value is calculated based on the activity type.

Another embodiment of the invention discloses a system for generating health data including a network server configured to store a data set having a set of time series profiles of a body parameter of a user associated with a corresponding set of activity types of the user, such that each time series profile is associated with a corresponding activity type a sensor configured to measure the body parameter of a user over an interval of time to form a time series profile of the body parameter over the interval of time; and a processor configured for: matching the time series profile against the a set of time series profiles of the data set; selecting, from the stored data set, the activity type associated with the matched time series profile; and calculating a value associated with a health metric based on the activity type.

Yet another embodiment discloses a non-transitory computer-readable storage medium, having embodied thereon a program executable by a processor to perform a method for generating health data. The method includes receiving a sensor data set sensed by one or more sensors on the wearable device, the sensor data set indicating a time series profile a body parameter of a user of the wearable device over a duration of an activity having an activity type and performed by the user of the wearable device; determining a metric method matching the time series profile, the metric method is configured for calculating a health metric of the user performing the activity having the activity type; and calculating a value of the health metric using the metric method.

Therefore, a need in the art to increase the accuracy of values for metrics generated by wearable devices, and a need to more accurately identify or discern a type of activity, which may then be correlated to a basis for a wearable device calculation, are achieved.

Some embodiments of the invention are based on insight that by more accurately identifying a given activity, a more appropriate algorithm or calibration tool may be utilized in the context of that activity thereby increasing the functionality and benefits of the wearable device.

### BRIEF DESCRIPTION OF THE DRAWINGS

FIG. 1A illustrates a network environment in which an exemplary system for movement-type calibration of wearable devices may be implemented according an embodiment of the present invention.

FIG. 1B illustrates exemplary data collected from exemplary sensors of the wearable device according an embodiment of the present invention.

FIG. 2 illustrates exemplary devices and algorithms that may be used in a system for movement-type calibration of wearable devices according an embodiment of the present invention.

FIG. 3 illustrates exemplary sets of sensor data that may be sensed during different activities by a system for movement-type calibration of wearable devices according an embodiment of the present invention.

FIG. 4 shows a flowchart illustrating an exemplary calibration method for movement-type calibration of wearable devices according an embodiment of the present invention.

FIG. 5 shows a flowchart illustrating an exemplary matching method for movement-type calibration of wearable devices according an embodiment of the present invention.

FIG. 6 illustrates a mobile device architecture that may be utilized to implement the various features and processes described herein according an embodiment of the present invention.

FIG. 7 shows a flowchart illustrating an exemplary calculation method for movement-type calibration of wearable devices according an embodiment of the present invention.

FIG. 8 shows a block diagram of a computer-implemented method for generating health data according to one embodiment of the invention.

FIG. 9 shows a schematic of the stored data set according to one embodiment of the invention.

FIG. 10 shows a schematic of training the regression function according to one embodiment of the invention.

FIG. 11A shows an example of the stored data set that includes references to metric methods for calculating the health metric according to one embodiment of the invention.

FIG. 11B shows an example of the stored data set of alternative embodiment.

FIG. 12 shows a block diagram of a computer-implemented method for generating health data according to another embodiment of the invention.

### DETAILED DESCRIPTION

Embodiments of the present invention may include reviewing data stored in a library of information that stores sensor data accumulated for a plurality of different types of activity. The information in the library may be compared to data sensed by sensors at or near a wearable device. The data sensed may be stored in a memory, and the sensed data may be compared to the information in the library when identifying an activity type that corresponds to data sensed by sensors.

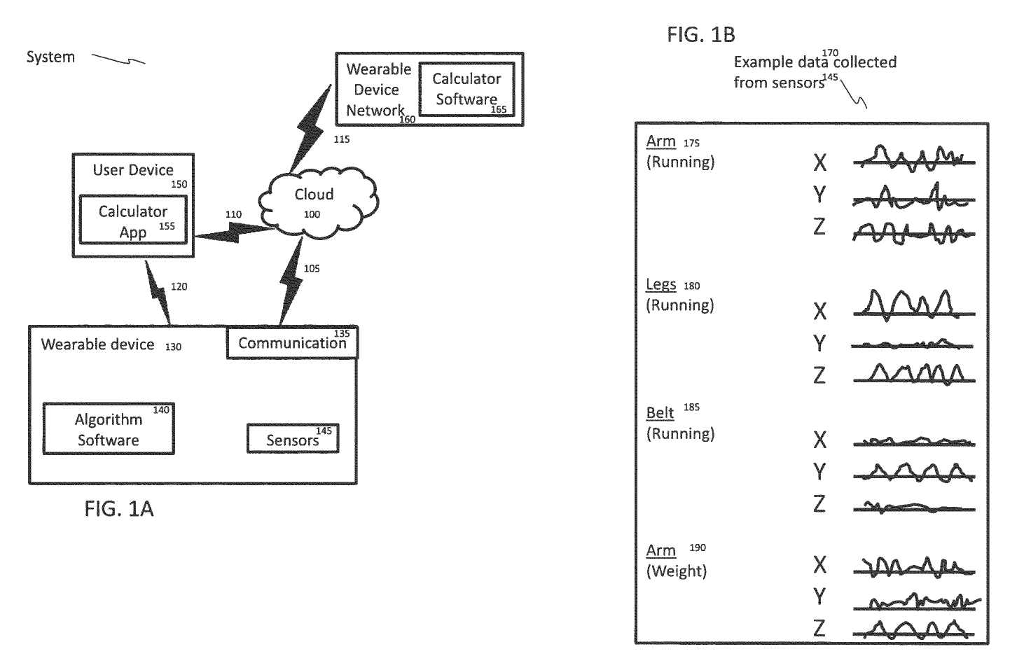

FIG. 1A illustrates a network environment in which an exemplary system for movement-type calibration of wearable devices may be implemented. The network environment may include a wearable device 130 in communication with a user device 150 (either directly through a connection 120 or through the cloud/internet 100 using a connection 105 and a connection 110), and a wearable device network 160 with one or more servers that is connected to the internet/cloud 100 (connection 115).

The wearable device 130 may include sensors 145, algorithm software module 140, and a wired and/or wireless communication interface 135 (e.g. a USB port module, a

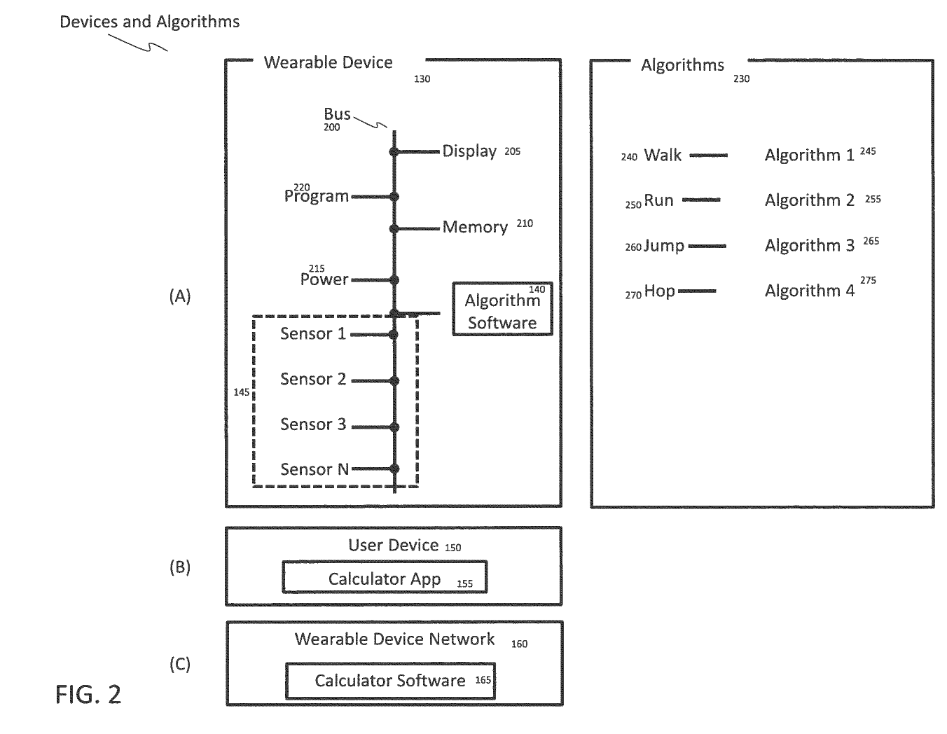

FireWire port module, a Lightning port module, a Thunderbolt port module, a Wi-Fi connection module, a 3G/4G/LTE cellular connection module, a Bluetooth connection module, a Bluetooth low energy connection module, a, Bluetooth Smart connection module, a near field communication module, a radio wave communications module). The algorithm software module 140 may be stored in a wearable device memory 210 (see FIG. 2) and executed by a wearable device processor (not shown). The components and elements of the wearable device 130 as depicted in FIG. 1A should be interpreted as illustrative rather than limiting; a wearable device 130 need not include all of these components and/or may include additional components not listed herein.

These sensors 145 of the wearable device 130 may include, for example, sensors measuring hydration, calories, blood pressure, blood sugar or glucose, insulin, body temperature (i.e., thermometer), heat flux, heart rate, weight, sleep, number of steps (i.e., pedometer), velocity or acceleration (i.e., accelerometer), vitamin levels, respiratory rate, heart sound (i.e., microphone), breathing sound (i.e., microphone), movement speed, skin moisture, sweat detection, sweat composition, nerve firings (i.e., electromagnetic sensor), or similar health measurements.

The user device 150 may include a calculator application 155 and a wire and/or wireless communication interface (e.g. a USB port module, a FireWire port module, a Lightning port module, a Thunderbolt port module, a Wi-Fi connection module, a 3G/4G/LTE cellular connection module, a Bluetooth connection module, a Bluetooth low energy connection module, a Bluetooth Smart connection module, a near field communication module, a radio wave communications module). The calculator application 155 may be stored in a user device memory (not shown) and executed by a user device processor (not shown). The components and elements of the user device 150 should be interpreted as illustrative rather than limiting; a user device 150 as depicted in FIG. 1A need not include all of these components and/or may include additional components not listed herein.

In one embodiment, the user device 150 may be, for example, a smartphone, a tablet, a laptop computer, a desktop computer, a gaming console, a smart television, a home entertainment system, a second wearable device, or another computing device.

The wearable device network 160 may include one or more servers. One of more of the wearable device network 160 servers may execute, using a processor, a calculator software module 165. The servers of the wearable device network 160 may also include a wired and/or wireless communication interface (e.g. a USB port module, a FireWire port module, a Lightning port module, a Thunderbolt port module, a Wi-Fi connection module, a 3G/4G/LTE cellular connection module, a Bluetooth connection module, a Bluetooth low energy connection module, a, Bluetooth Smart connection module, a near field communication module, a radio wave communications module). The components and elements of the wearable device network 160 as depicted in FIG. 1A should be interpreted as illustrative rather than limiting; a wearable device network 160 need not include all of these components and/or may include additional components not listed herein.

FIG. 1B illustrates exemplary data 170 collected from exemplary sensors 145 of the wearable device 130. As illustrated, the sensors 145 on the wearable device worn by a person may include a time series profile of measurements corresponding to the activity of a user's arms 175, legs 180, and belt 185 (e.g., belly or torso) when running (175, 180, 185) and information corresponding to the activity of a person's arms 190 when weight lifting 190. The data may pertain to movement in three dimensions (X, Y, and Z) as sensed by an acceleration sensor among the sensors 145. Movements of the user of the wearable device 130 in the X, Y, and Z directions may be characterized by sets of graphs (or signals) varying over time, as depicted corresponding to each body part (175, 180, 185, 190) in FIG. 1B. Notice that each body part is associated with sensor data (of the sensors 145) that is different for each body part and associated sensor and associated set of X/Y/Z graphs. Even though three of the four sets of X/Y/Z graphs recorded when a person was running (i.e., 175, 180, 185), each set of X/Y/Z graphs was sensed by a sensor at a different body part (e.g., arms 175, legs 180, and belt 185), thereby yielding different results for each body part and associated sensor. Sensors used in movement-type calibration are not limited to acceleration sensors, but may be any sensor capable of recording parametric information regarding a person's body. For example, sensors may be heat sensors or sensors that sense the movement or changes of heat (heat flux).

A library of information for identifying an activity type (e.g., walking, running, lifting weights, walking with weights, running with weights, jumping, hopping, jumping rope, squatting, swimming, climbing, skiing, snowboarding, skateboarding, bicycling, stretching, doing gymnastics, doing yoga, or playing a sport) may include information stored at one or more servers of the wearable device network 160, at the user device 150, or at the wearable device 130. Such information may be provided to the wearable device 130 from a generic library of pre-recorded activity types. Alternatively, a user of the wearable device 130 may record his/her own personal sensor data 170 from the sensors 145 for a designated activity type (e.g., through a combination of readings from sensors 145 and a graphical user interface or “GUI”) and uploaded to the library for storage.

Based on the sensor data 170 and algorithms (at algorithm software module 140 or calculator app 155 or calculator software module 165) running on a processor (of the wearable device 130 or of the mobile device 150 or of a server of the wearable device network 160), the processor may calculate work or effort expended by a person during an activity. Measures of work or effort expended by the person may include, yet are not limited to number of calories burned, amount of heat generated, number of steps stepped, pace, and repetition rate. Each activity type may be associated with a different algorithm (see FIG. 2) for calculating the measures of work or effort that is tailored to the particular activity. Each specific new algorithm may be derived, by the wearable device 130 or by the mobile device 150 or by the wearable device network 160, using mathematic and scientific principles applied to the raw measurements from sensors 145. By measuring and mapping similar activities performed by different individuals and correlating those activities to determine calories burned (or other measures) during those activities, new algorithms 230 can be generated that correspond to new activity types over time.

The algorithms or metric methods 230 that calculate the measures of work or effort may be run on a processor in the wearable device 130, in the user device 150, or in the wearable device network 160. In certain instances, the wearable device 130 may transmit sensor data 170 from sensors 145 to the user device 150 (either directly through a connection 120 or through the cloud/internet 100 using a connection 105 and a connection 110) or to the wearable device network 160 (through the cloud/internet 100 using a connection 105 and a connection 115, or using the user device 150 as a proxy and thus transmitting through connection 120 to connection 110 to connection 115). Once the sensor data 170 from the sensors 145 is received at the user device 150 or wearable device network 160, the calculator application 155 in the user device 150 or the calculator software module 165 in the wearable device network 160 may calculate the measures of work or effort based on an algorithm of a set of algorithms 230 (see FIG. 2) selected to correspond to the activity indicated by the sensor data 17 of the sensors 145.

In certain instances, the wearable device 130 may include one or more different sensors 145 communicating using wireless data communication. Any wireless data transmission technology standard in the art (e.g., Bluetooth™ or cellular data communications) may be used. In certain instances, the sensors may communicate sensor data 170 to the user device 150 using Bluetooth™ (e.g., connection 120), and the user device 150 may then communicate the sensor data (or calculated measures of work) to the wearable device network 160 using a cellular signal (i.e., through connection 110 and connection 115), or vice versa. In certain embodiments, each sensor of the sensors 145 may be an independent sensor that is not physically connected to another sensor; in other embodiments, each of the sensors 145 may be collectively or partially connected to each other.

FIG. 2 illustrates exemplary devices (130, 150, 160) and algorithms or methods 230 that may be used in a system for movement type calibration of wearable devices 130. Such devices may include a wearable device 130, a user device 150, and a wearable device network server 160. In one embodiment, the wearable device 130 may include a display 205, a memory 210, a power supply 215 (e.g., a rechargeable or non-rechargeable battery), an algorithm software module 140, and sensors 1-N (145). In one embodiment, each of these components and elements is connected together with a single communication bus 200; in other embodiments, the wearable device 130 may be connected using a more divergent approach, for instance by including a subset of the sensors 145 that are connected to a second bus (not shown) and connected wirelessly to the bus 200. The components and elements of the wearable device 130 as depicted in FIG. 2 should be interpreted as illustrative rather than limiting; a wearable device 130 need not include all of these components and/or may include additional components not listed herein.

As shown in FIG. 1, the user device 150 may include a calculator application 155, and the wearable device network 160 may include calculator software module 165. The components and elements of the user device 150 and the wearable device network 160 as depicted in FIG. 2 should be interpreted as illustrative rather than limiting; the user device 150 and the wearable device network 160 need not include all of these components and/or may include additional components not listed herein.

The wearable device (or one of the other devices) may further include algorithm software module 140 for any of a plurality of available algorithms related to different activities. Algorithms 230 depicted in FIG. 2 include algorithm 1 245, algorithm 2 255, algorithm 3 265, and algorithm 4 275, each respectively corresponding to a different activity (e.g., algorithm 1 245 for walking 240, algorithm 2 255 for running 250, algorithm 3 265 for jumping 260, and algorithm 4 275 for hopping 270). The sensors 145 depicted in FIG. 2 may include sensors capable of measuring body activity, such as acceleration, heat, heat flow (flux), moisture, hydration, calories, blood pressure, blood sugar or glucose, insulin, body temperature (i.e., thermometer), heart rate, weight, sleep, number of steps (i.e., pedometer), velocity or acceleration (i.e., accelerometer), vitamin levels, respiratory rate, heart sound (i.e., microphone), breathing sound (i.e., microphone), movement speed, skin moisture, sweat detection, sweat composition, nerve firings (i.e., electromagnetic sensor), or similar health measurements. Displays on the wearable device 130 may be a liquid crystal display (LCD), a series of light emitting diodes (LEDs), lights, an organic light emitting diode display (OLED), an electronic paper display (e.g., gyricon, electrophoretic, electrofluidic, or electrochromic displays), or another display screen of any type known in the art. The algorithm software module 140 may be run on a processor (not shown), a state machine in a field programmable gate array (FPGA), or an application specific integrated circuit (ASIC).

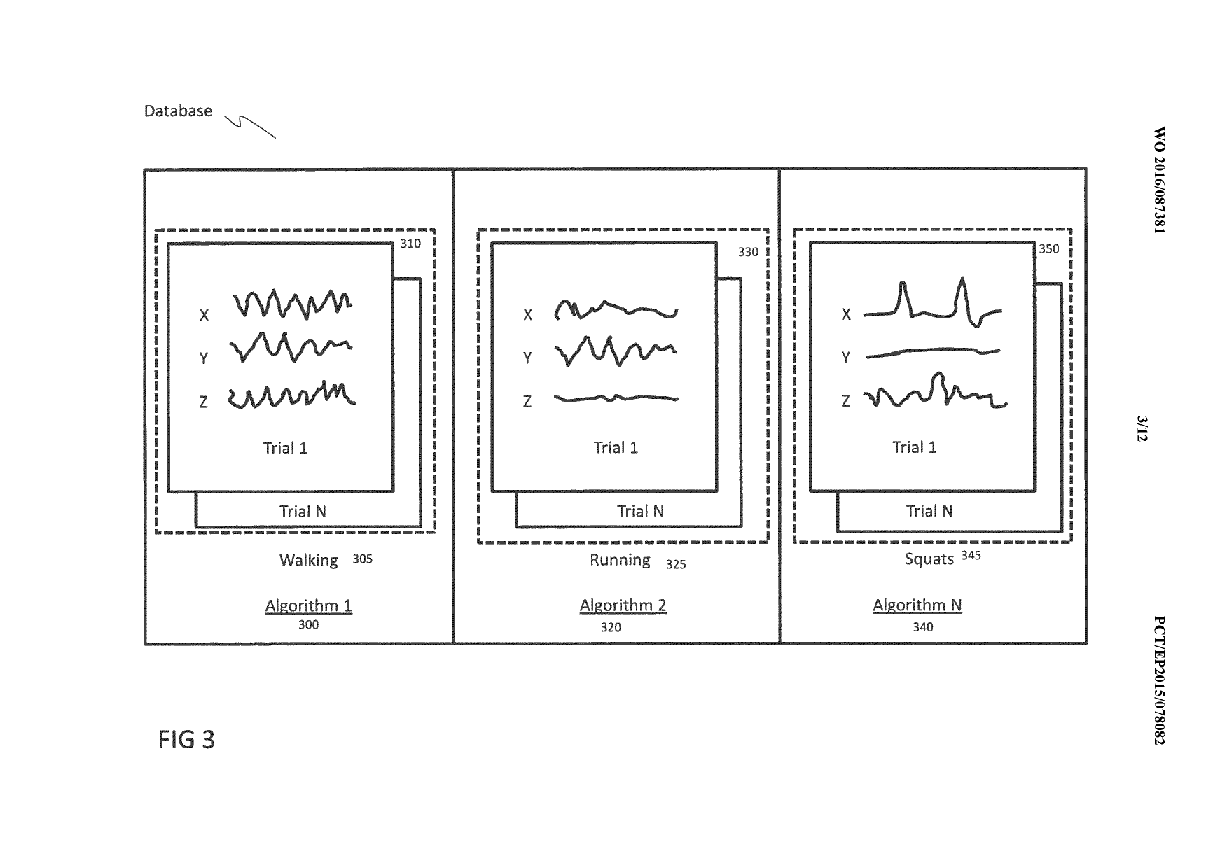

FIG. 3 illustrates exemplary sets of sensor data that may be sensed during different activities by a system for movement type calibration of wearable devices. Data may be recorded during a series of trials (310, 330, 350) (e.g., trials 1-N) for each activity type (305, 325, 345). Activity types illustrated in FIG. 3 include walking 305, running 325, and squats 345. Each set of data may be associated with a different algorithm (e.g., walking 305 is associated with algorithm 1 300, running 325 is associated with algorithm 2 320, and squats 345 is associated with algorithm 3 340). The sensors may sense acceleration, heat, heat flow (flux), moisture, or other parameters associated with a person's body as discussed previously. These parameters may be measured from a plurality of trials (e.g., as in 310, 330, and 350) from one or more persons. Each movement type (305, 325, 345) is thus associated with an algorithm (300, 320, 340) and with a set of sensor measurement trials 1-N (310, 330, 350). Although each set of trials (310, 330, 350) is marked as including N trials (“1-N”), it should be noted that each set of trials may include one or more trials, and that each set of trials may include a different number of trials. Each trial (e.g., Trial 1) may include sensor data from one or more of the sensors 145; for example, each trial may include X/Y/Z coordinate data measured during the movement using a location sensor or accelerometer of the sensors 145.

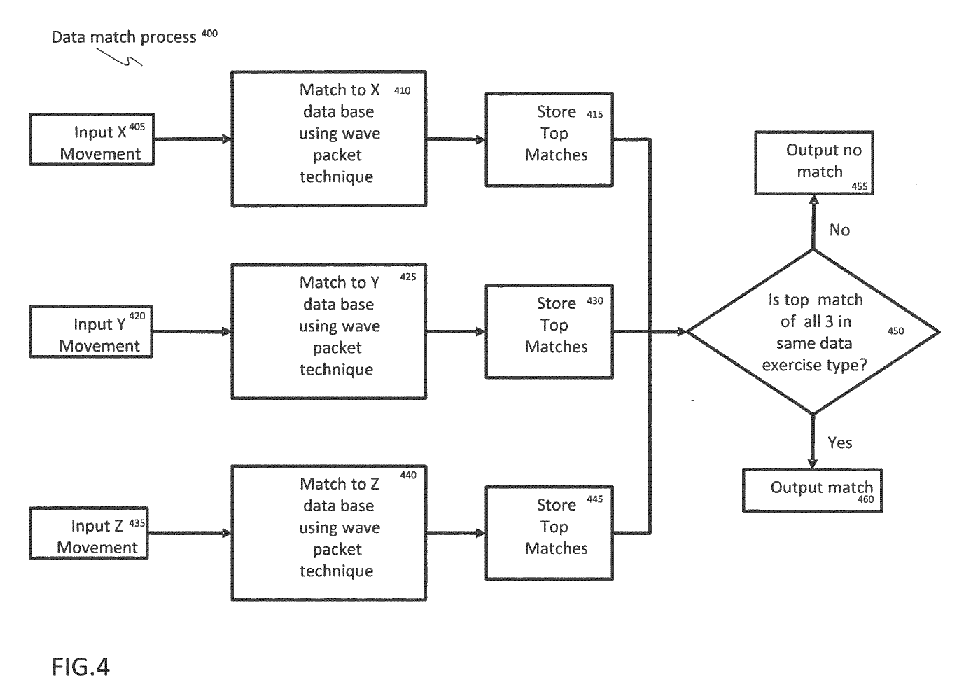

FIG. 4 is a flowchart illustrating an exemplary matching method 400 for movement type calibration of wearable devices 130. Three different types of movement (e.g., X, Y, and Z movements) may be sensed by one or more sensors 145 and characterized by sensor data 170. Accordingly, the exemplary process 400 may include inputting an X movement (block 405), inputting a Y movement (block 420), and inputting a Z movement (block 435). Each set of sensor data may be matched by comparison to data sets in databases X, Y, and Z data using a wave packet technique. Accordingly, the X movement may be matched to the X database (block 410), the Y movement may be matched to the Y database (block 425), and the Z movement may be matched to the Z database (block 440). Top matches may be identified and stored for each set. Accordingly, top matches may be identified and stored for the X movement set (block 415), for the Y movement set (block 430), and for the Z movement set (block 445). It may then be determined whether the three sets of sensed data matches previously stored data sets corresponding to a particular exercise type (block 450). The step described in block 450 may determine, for example, whether the top match corresponding to the “X movement” sensor data set, the top match corresponding to the “Y movement” sensor data set, and the top match corresponding to the “Z movement” sensor data set all correspond to the same exercise type (e.g., walking, running, lifting weights, walking with weights, running with weights, jumping, hopping, jumping rope, squatting, swimming, climbing, skiing, snowboarding, skateboarding, bicycling, stretching, doing gymnastics, doing yoga, or playing a sport). When the determination step indicates a match, the match may be output as a result (e.g., the user is/was most likely running) (block 460). When the determination step does not indicate a match, an indication of no match may be output (i.e., the user's activity cannot be determined) (block 455). In certain embodiments, the sensor may sense an acceleration, a weight, or another parameter as described in relation to previous figures.

After the determination step (block 450) indicates a match (block 455), further calculations may be performed given the knowledge of the activity type that the user was performing. For instance, once the electronic device (i.e., the wearable device 110, the user device 150, and/or the wearable device 160) understands that the user was performing a particular activity (e.g., running), the electronic device can calculate a health metric (e.g., calories burned) with increased accuracy due to an understanding of the type of activity being performed by the user of the wearable device. As used herein, the health metric can be any metric and/or value expressing the health status of the user. For example, according to one embodiment, the health metric is common to multiple activity types (e.g., total calories burned, average calorie burn rate, average change in calorie burn rate over time) rather than an “individualized” health metric that is particular to a specific activity type (e.g., steps walked, steps ran, squats performed, distance walked, distance ran, laps swam, elevation climbed, weight lifting repetitions). Thus, in one embodiments, the health metric is calculated specifically not an “individualized” health metric particular to a specific activity type or to several activity types (e.g., steps walked and ran, distance walked and ran). In another embodiment, the health metric may be an “individualized” health metric.

In some embodiments, the process of FIG. 4 may be performed using additional sensor types. In particular, while the exemplary process of FIG. 4 illustrates input of X/Y/Z location or movement sensors, a different embodiment could include different sensor data set types. For example, a different embodiment could take into account data sets from a Z movement sensor and a pulse sensor, which could then decide what activity the user is performing by comparing to Z movement data sets as well as pulse data sets. The health metric that is ultimately calculated could then be based on all of these sensor data sets and/or other sensor data sets; for instance, an electronic device could base a “total calories burned” health metric on both the accelerometer (e.g. Z movement sensor) data set as well as the pulse data set, and may also base it on a third sensor (e.g., a blood pressure sensor).

Once a value of the health metric has been calculated, in some embodiments, it may be stored by the wearable device 130, the user device 150, and/or the wearable device network 160. In some embodiments, the value may be output to the user at display 205 of the wearable device 130, or at a display of the user device 150. In some embodiments, once a match is determined (block 460), the matching activity type can be displayed to the user at display 205 of the wearable device 130, or at a display of the user device 150, and the user may be presented with a user interface (e.g., of the wearable device 130 or of the user device 150) in which he/she can correct the activity type if it has been incorrectly determined.

In some embodiments, where no match has been found (block 455), a user may be allowed, through a user interface (e.g., of the wearable device 130 or of the user device 150), to input a new activity type (e.g., lifting heavy boxes), and in some cases, to input a health metric calculation algorithm or a notification setting.

While the flow chart in FIG. 4 shows a particular order of operations performed by certain embodiments of the invention, it should be understood that such order is exemplary (e.g., alternative embodiments can perform the operations in a different order, combine certain operations, overlap certain operations, etc.).

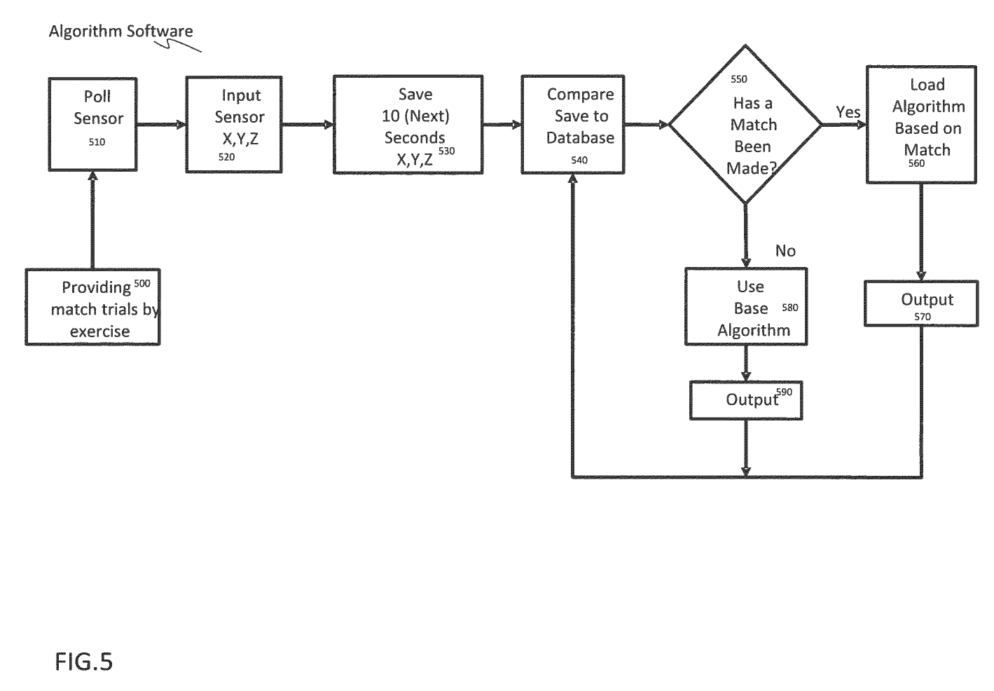

FIG. 5 is a flowchart illustrating an exemplary calibration method for movement type calibration of wearable devices 130. In step 500, exercise data from a series of match trials may be provided to a library. In step 510, one or more sensors may be polled for data periodically over a stretch of time. In step 520, the polled sensor data (e.g., including X, Y, and Z acceleration components) may be input into an electronic device (e.g., the wearable device 130, the user device 150, or a server of the wearable device network 160) running algorithms consistent with the present invention. The algorithms may be run in a wearable device 130, in a user device 150, or in a wearable device network 160. In step 530, the X, Y, and Z new sensed data (e.g., spanning 10 seconds) may be stored in a memory (e.g., of the wearable device 130, of the user device 150, or of the wearable device network 160). In step 540, the new sensed data may be compared with the data stored in the database library. As noted, the comparison and matching may be based on wave packet structure.

In step 550, it may be determined whether a match has been made. When the new sensed data matches a data set stored in the database library, the method may proceed to step 560 where an algorithm consistent with the match may be loaded into memory for execution. In step 570, the result calculations from step 560 may be output (e.g., to display 205 of wearable device 130 or to a display of user device 150, or through a speaker at wearable device 130 or user device 150). Next, the method may return to step 540. The algorithm loaded in step 560 may be developed using tests from a series of trials. The algorithm may be specific to a particular type of exercise.

If, after the comparison (step 540) is concluded in step 550, a match has not been made, a base algorithm may be used to perform calculations on the sensed data (step 580). For example, this base algorithm may be a “generic” calorie calculation based on the sensor data and not modified by an activity type. In step 590, the result of calculations from step 580 may be output (e.g., to display 205 of wearable device 130 or to a display of user device 150, or through a speaker at wearable device 130 or user device 150). After step 590, the method may revert to step 540 in order to make more data comparisons.

In some embodiments, use of a base algorithm (step 580) may be replaced or supplemented with an additional step, where the wearable device 130 or user device 150 receives an input from the user through a user interface, the input allowing the user to select an activity type (e.g., from a list or grid or textual input) and then loads an algorithm based on the selected activity type, or allows the user to customize an algorithm for a new activity type. Similarly, if a match has been made (step 550 to step 560), an input can be received from a user interface (e.g., from wearable device 130 or user device 150) confirming the matched activity type or selecting a replacement activity type prior to loading an algorithm (step 560). Further, in some embodiments, outputs (570 or 590), or user interface interactions, may be accompanied by an alert, such as a vibration, a sound, a graphic, a video, an indicator light, or some other type of alert presented by the wearable device 130 (e.g., using display 205) or user device 150.

While the flow diagram in FIG. 5 shows a particular order of operations performed by certain embodiments of the invention, it should be understood that such order is exemplary (e.g., alternative embodiments can perform the operations in a different order, combine certain operations, overlap certain operations, etc.).

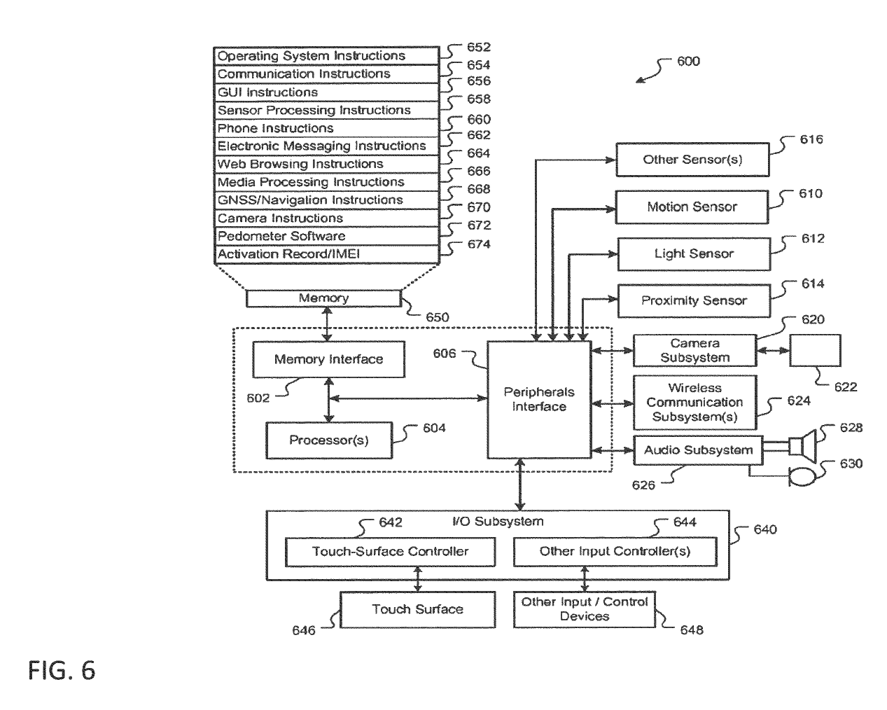

FIG. 6 illustrates a mobile device architecture that may be utilized to implement the various features and processes described herein. Architecture 600 can be implemented in any number of portable devices including but not limited to smart phones, electronic tablets, and gaming devices. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 are meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.5 or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.5 communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VXWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; pedometer software 672 to facilitate pedometer-related processes; activation record/IMEI software 674 to facilitate activation record/IMEI-related processes and other instructions 676 for any other application that may be operating on or in conjunction with the mobile computing device. Memory 650 may also store other software module instructions for facilitating other processes, features and applications, such as applications related to navigation, social networking, location-based services or map displays.

Various embodiments described herein achieve various functionality through the execution of instructions by a processor. It will be understood that, while various examples are described in the context of instructions actively performing steps or other actions, any such actions will actually be performed by the processor that executes such instructions.

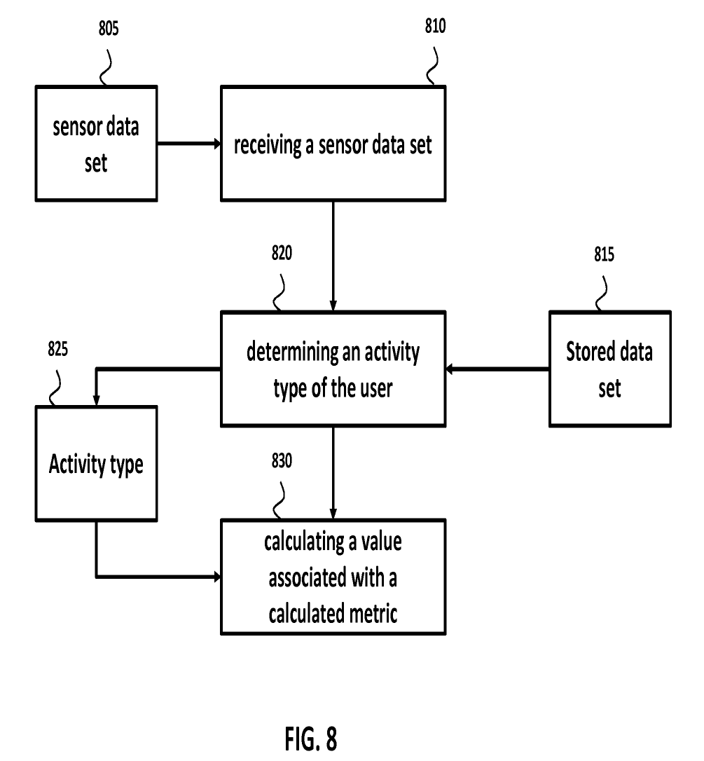

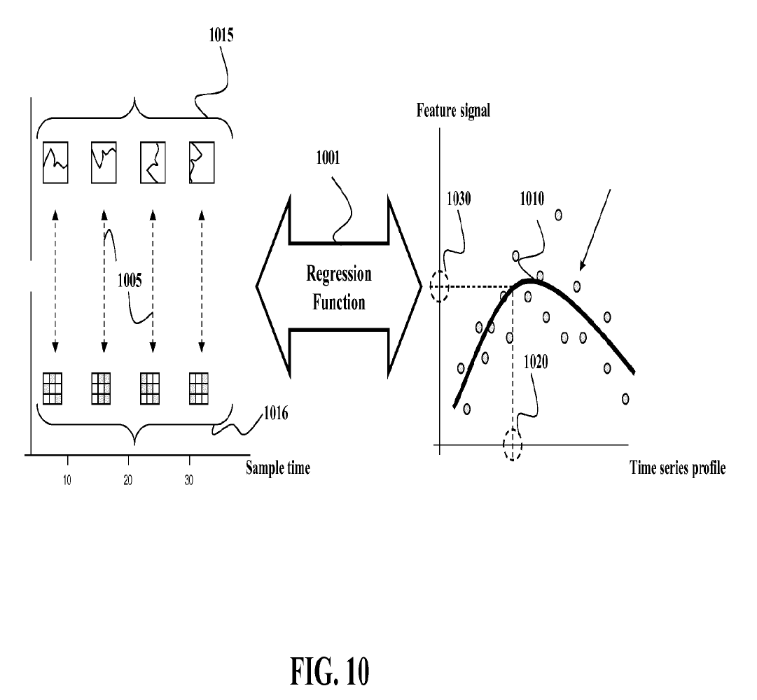

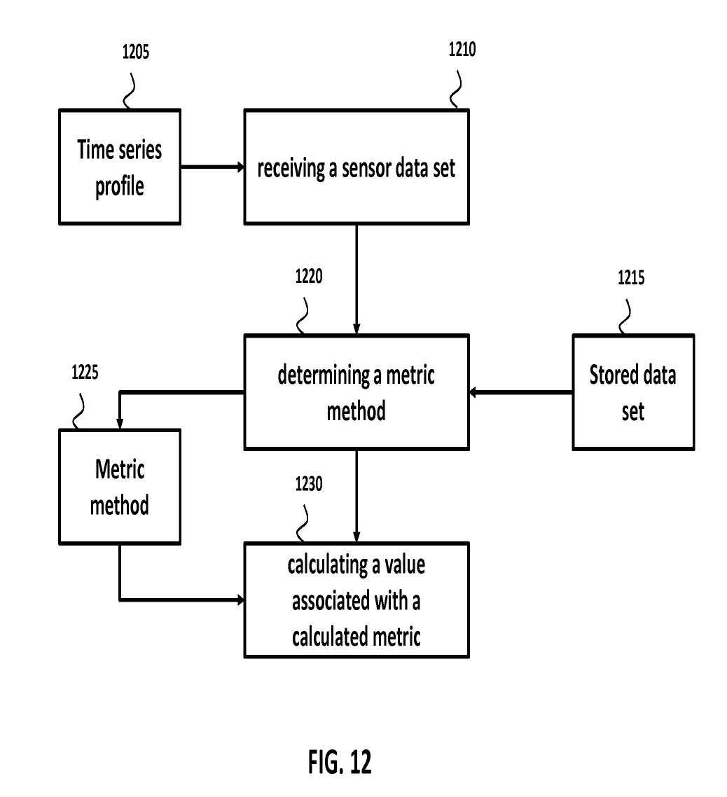

Note that the calculator app 155, the calculator software 165, the Algorithm software 140, the Algorithm 230, the Algorithm 1 (245, 300), the Algorithm 2 (255, 320), the Algorithm 3 (265, 340), the Algorithm 4 275, the data match process 400, the calibration method for movement type calibration of wearable devices, the pedometer software 672, the activation record/IMEI software 674, the computer-implemented method for generating health data as depicted in FIG. 8, the schematic of training the regression function as depicted in FIG. 10, and the a computer-implemented method for generating health data as depicted in FIG. 12 are software that are stored in one of the memory for execution by the processor 604.

The memory 650 may store operating system instructions 652, communication instructions 654, GUI instructions 656, sensor processing instructions 658, phone instructions 660, electronic messaging instructions 662, web browsing instructions 664, media processing instructions 666, GNSS/navigation instruction 668, camera instructions 670, and other instructions 676 for execution by the processor 604. It will be understood that these instructions may be alternatively or additionally stored in a non-volatile storage device such as the storage device storing the reference link database or another storage device (not shown). For example, the instructions may be stored in a flash memory or an electronic read only memory (ROM) until they are to be executed by the processor, at which point they are copied to the memory 650. As used herein, the term storage will be understood to refer to non-volatile memories.

The processor 604 may be virtually any device capable of performing the functions described herein including the functions described above in connection with the operating system instructions 652, communication instructions 654, GUI instructions 656, sensor processing instructions 658, phone instructions 660, electronic messaging instructions 662, web browsing instructions 664, media processing instructions 666, GNSS/navigation instruction 668, camera instructions 670, and other instructions 676. For example, the processor 604 may include one or more microprocessors, one or more field-programmable gate arrays (FPGA), or one or more application-specific integrated circuits (ASIC). In some embodiments, the processor may not utilize stored instructions to perform some or all of the functions described herein; for example, an ASIC may be hardwired to perform one or more of the functions describe above with reference to the operating system instructions 652, communication instructions 654, GUI instructions 656, sensor processing instructions 658, phone instructions 660, electronic messaging instructions 662, web browsing instructions 664, media processing instructions 666, GNSS/navigation instruction 668, camera instructions 670, and other instructions 676. In some such embodiments, the operating system instructions 652, communication instructions 654, GUI instructions 656, sensor processing instructions 658, phone instructions 660, electronic messaging instructions 662, web browsing instructions 664, media processing instructions 666, GNSS/navigation instruction 668, camera instructions 670, and other instructions 676 may be omitted because they are already embodied in the processor 604 without the need for stored instructions.

Each of the above identified instructions and applications can correspond to a set of instructions for performing one or more functions described above. These instructions need not be implemented as separate software module programs, procedures, or modules. Memory 650 can include additional or fewer instructions. Furthermore, various functions of the mobile device may be implemented in hardware and/or in software module, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software module code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer may employ to access functions supporting the API. In some implementations, an API call can report to an application the capabilities of a device running the application, such as input capability, output capability, processing capability, power capability, and communications capability.

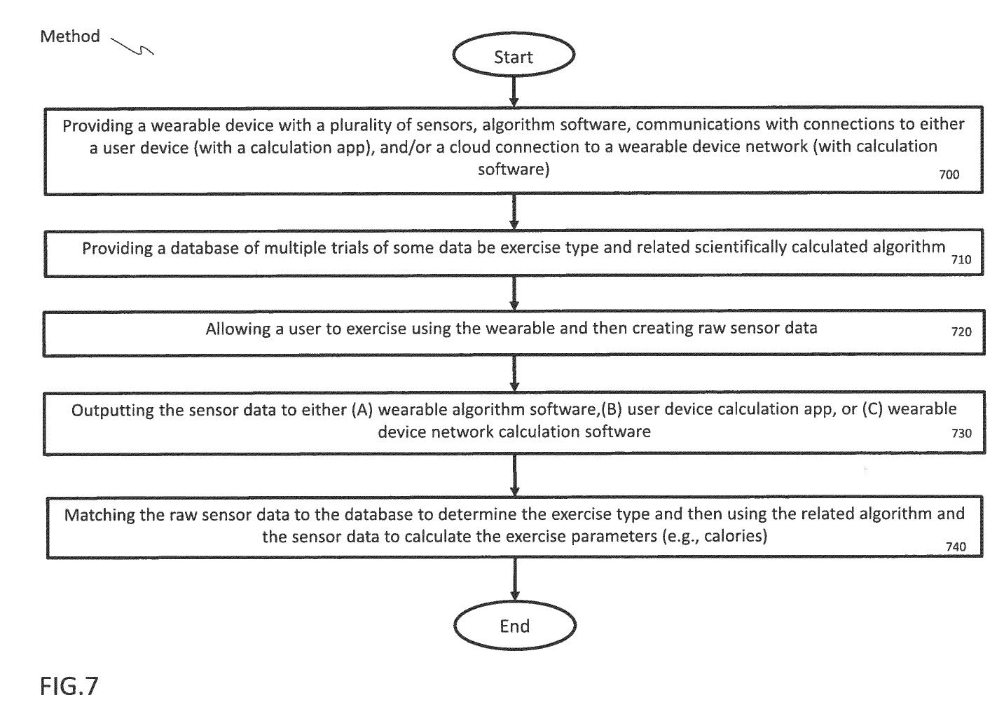

FIG. 7 depicts a flow chart of a method of the present invention. A wearable device 130 may be provided with a plurality of sensors 145, algorithm software module 140, communications interfaces 135 for connecting to a user device 150 (e.g., which may have a calculator app 155), and/or via the cloud/internet 100 to a wearable device network server 160 (e.g., which may have calculation software module 165) (block 700).

A database may be provided for storing sensor data 170 regarding multiple trials categorized by exercise type and associated with one or more algorithms 230 (block 710). Access to a database library may be provided to the wearable device 130 that is performing calculations on sensor data 170.

A user may exercise while wearing the wearable device 130, which may then generate raw sensor data 170 through sensors 145 (block 720). The sensor data 170 may be output to the wearable device 130 (e.g. to be processed by algorithm software module 140), the user device 150 (e.g., to be processed by the calculation app 155), and/or wearable device network server 160 (e.g., to be processed by the calculation software module 165) (block 730). The raw sensor data may be compared and/or matched to data in the database to determine a match with any of the available predetermined exercise types (block 740). Related algorithms and the sensor data may further be used to calculate various exercise parameters (e.g., calories) (block 740). The sensed and matched data may be processed using an algorithm based on the matching exercise type. Such processing may provide various measures of work or effort.

While the flow diagram in FIG. 7 shows a particular order of operations performed by certain embodiments of the invention, it should be understood that such order is exemplary (e.g., alternative embodiments can perform the operations in a different order, combine certain operations, overlap certain operations, etc.).

FIG. 8 shows a block diagram of a computer-implemented method for generating health data according to one embodiment of the invention. Steps of the method can be performed by a processor of a wearable device, a processor of a network server, or a combination thereof. The method receives 810 a sensor data set 805 measured by one or more sensors of a wearable device over an interval of time. The sensor data set indicates a time series profile of a body parameter of a user of the wearable device over the interval of time. As a matter of definitions, a time series is a sequence of successive data points made over the time interval. As used herein, the time series profile is a function of the successive measurements of one or more sensors of the wearable device.

The method determines 820 an activity type 825 of the user matching the time series profile and calculates 830 a value associated with a health metric, wherein the value is calculated based on the activity type. For example, in one embodiment, the method compares the sensor data 805 with the stored data set 815 to determine the matching activity type of the user.

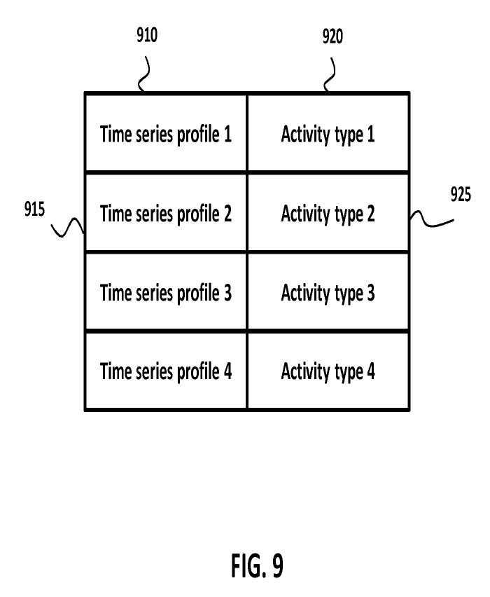

FIG. 9 shows a schematic of the stored data set 815 according to one embodiment of the invention. In this embodiment, the stored data set includes a set of time series profiles 910 of a body parameter of a user associated with a corresponding set of activity types of the user 920. In this example, each time series profile 915 is associated with a corresponding activity type 925. In such a manner, the activity type associated with the time series profile of the body parameter can be retrieved.

To facilitate the matching of the time series profiles, some embodiment extract feature signals from the row sensor data. Such feature signals can be more efficiently stored and compared. Examples of the feature signal include a waveform, appearance and statistics based descriptors, pixel intensities, intensity histograms, histogram of oriented gradients (HoGs), feature covariance descriptors, first and higher order region statistics, principal components or independent components of the measurements of the body parameter, frequency transforms, e.g., Fourier, discrete cosine, and wavelet transforms, and eigenfunctions.

Some embodiments of the invention are based on understanding that it is not always practical to expect a perfect match between the measured time series profile and the stored time series profile. For example, the time series profile associated with running activity of the user can have multiple variations due to differences in the running style. Accordingly, some embodiments of the invention use regression analysis as a statistical process for estimating the relationships between the measured and the stored time series profiles. For example, one embodiment of the invention trains a regression function establishing relationship between the time series profile and the feature signals of the stored data set.

FIG. 10 shows a schematic of training 1001 the regression function 1010. The regression function establishes a correspondence 1005 between the time profile series 1015 and the set of the feature signals 1016. Knowing the regression function 1010, the particular feature signal 1030 can be determined from the particular time series profile 1020. The feature signals can be of any dimensions. The regression function 1010 can be any complex function. For example, the recreation function can be linear, nonlinear, and nonparametric regression function. The regression function can be a polynomial function or a spline.

In some embodiments of the invention, each activity type of the user is associated with a metric method for calculating the health metric. To that end, for at least two different activity types of the user are associated with two different metric methods that are configured and used for determining the value of the health metric. Examples of the metric method are provided in relation to FIGS. 2 and 3. In some embodiments, the stored data set 815 includes references to the metric method.

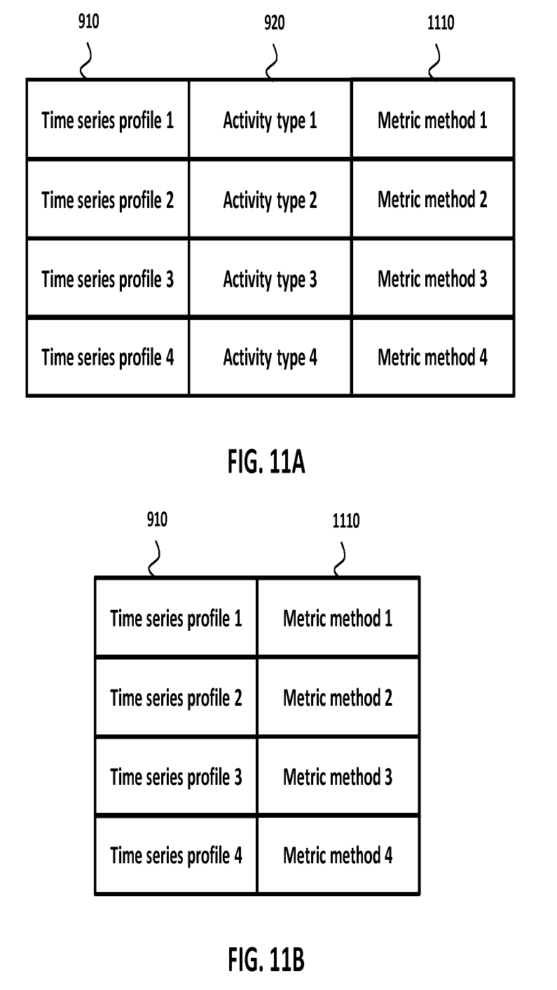

FIG. 11A shows an example of the stored data set that includes references 1110 to metric methods for calculating the health metric according to one embodiment of the invention. In this embodiment, the stored data set connects the time series profiles with the corresponding activity type and connects the activity type with the corresponding metric method.

FIG. 11B shows an example of the stored data set of alternative embodiment. In this embodiment, the time series profiles 910 are directly associated with the metric methods 1110.

FIG. 12 shows a block diagram of a computer-implemented method for generating health data according to one embodiment of the invention that takes advantage of the direct correspondence between the time series profiles 910 and the metric methods 1110. The method receives 1210 the time series profile 1205 of the body parameter of the user. The method determines 1220 a metric method 1225 for calculating the health metric by matching the time series profile 1205 with stored time series profiles associated with the metric methods. Upon finding a match between received and stored time series profiles, the matric method 1225 associated with the matched time series profile is selected. The method calculates 1030 a value associated with a health metric using the selected metric method 1225.

Embodiments of the invention also relate to an apparatus for performing the operations herein. Such a computer program is stored in a non-transitory computer readable medium. A machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures can be performed by processing logic that comprises hardware (e.g. circuitry, dedicated logic, etc.), software module (e.g., embodied on a non-transitory computer readable medium), or a combination of both. Although the processes or methods are described above in terms of some sequential operations, it should be appreciated that some of the operations described can be performed in a different order. Moreover, some operations can be performed in parallel rather than sequentially.

While various embodiments have been described above, it should be understood that they have been presented by way of example only, and not limitation. The descriptions are not intended to limit the scope of the invention to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the invention as defined by the appended claims and otherwise appreciated by one of ordinary skill in the art. The scope of the invention should, therefore, be determined not with reference to the above description, but instead should be determined with reference to the appended claims along with their full scope of equivalents.

1. A computer-implemented method for generating health data, the method comprising:
receiving a sensor data set measured by one or more sensors of a wearable device over an interval of time, the sensor data set indicating a time series profile of a body parameter of a user of the wearable device over the interval of time;
determining an activity type of the user matching the time series profile; and
calculating a value associated with a health metric, wherein the value is calculated based on the activity type.

2. The method of claim 1, wherein the health metric is applicable to a plurality of activity types.

3. The method of claim 1, further comprising:
extracting a feature signal from the time series profile;
matching the extracted feature signal with a stored data set having a set of feature signals associated with a corresponding set of activity types, such that each feature signal in the set of feature signals is associated with a corresponding activity type from the set of activity types; and
selecting, from the stored data set, the activity type associated with a feature signal matching with the extracted feature signal.

4. The method of claim 3, wherein the feature signal includes one or combination of a waveform, an appearance or a statistic based descriptor, an intensity histogram, a histogram of oriented gradients, a feature covariance descriptor, a first or a higher order region statistic, a frequency transform, and an eigenfunction.

5. The method of claim 1, further comprising:
receiving a second sensor data set indicating a second time series profile of the body parameter of the user or a second body parameter of the user over the interval of time;
determining a subset of activity types matching the time series profile; and
selecting the activity type from the subset of activity types that matches the second time series profile.

6. The method of claim 1, wherein the activity type is one of walking, running, lifting weights, walking with weights, running with weights, jumping, hopping, jumping rope, squatting, swimming, climbing, skiing, snowboarding, skateboarding, bicycling, stretching, doing gymnastics, doing yoga, or playing a sport.

7. The method of claim 1, wherein the health metric is related to one of a number of calories burned, a rate of calorie burn, an acceleration of calorie burn, a deceleration of calorie burn, an amount of power expended, or an amount of work performed.

8. The method of claim 1, wherein the one or more sensors measure at least one of hydration, calories, blood pressure, blood sugar, blood glucose, insulin, body temperature, heat, heat flux, heart rate, weight, sleep, number of steps, velocity, acceleration, vitamin levels, respiratory rate, heart sound, breathing sound, movement speed, skin moisture, sweat detection, sweat composition, or nerve firings.

9. The method of claim 1, further comprising:
receiving an input defining the activity type; and
associating the activity type with the time series profile.

10. The method of claim 9, further comprising:
extracting a feature signal from the time series profile;
storing the extracted feature signal and the activity type in a stored data set having a set of feature signals associated with a corresponding set of activity types, such that each feature signal is associated with a corresponding activity type.

11. The method of claim 1, further comprising receiving the stored data set from a network server.

12. The method of claim 1, wherein the calculating comprises:
selecting a metric method from a set of metric methods for calculating the health matric based on the activity type; and
performing steps of the metric method for calculating the value of the health metric.

13. A system for generating health data, the system comprising:
a network server configured to store a data set having a set of time series profiles of a body parameter of a user associated with a corresponding set of activity types of the user, such that each time series profile is associated with a corresponding activity type;
a sensor configured to measure the body parameter of a user over an interval of time to form a time series profile of the body parameter over the interval of time; and
a processor configured for:
matching the time series profile against the a set of time series profiles of the data set;
selecting, from the stored data set, the activity type associated with the matched time series profile; and
calculating a value associated with a health metric based on the activity type.

14. The system of claim 13, wherein the health metric is applicable to a plurality of activity types.

15. A non-transitory computer-readable storage medium, having embodied thereon a program executable by a processor to perform a method for generating health data, the method comprising:
receiving a sensor data set sensed by one or more sensors on the wearable device, the sensor data set indicating a time series profile a body parameter of a user of the wearable device over a duration of an activity having an activity type and performed by the user of the wearable device;
determining a metric method matching the time series profile, the metric method is configured for calculating a health metric of the user performing the activity having the activity type; and
calculating a value of the health metric using the metric method.

16. (canceled)
